(19) United States
(12) Patent Application Publication
(10) Pub. No.: US 2022/0023573 A1
(43) Pub. Date: Jan. 27, 2022

(54) DROPLET ISOLATION ENDOSCOPY MASK

(71) Applicant: Board of Regents, The University of Texas System, Austin, TX (US)

(72) Inventors: Vicente A. Resto, Galveston, TX (US); Kathleen M. Kerr, Friendswood, TX (US)

(21) Appl. No.: 17/383,753

(22) Filed: Jul. 23, 2021

Related U.S. Application Data
(60) Provisional application No. 63/056,099, filed on Jul. 24, 2020.

(51) Int. Cl. A61M 16/06; A61M 16/10; A61M 16/20; A61M 16/08

(52) U.S. Cl. CPC A61M 16/0672; A61M 16/1065; A61M 16/0683; A61M 2202/0208; A61M 16/0816; A61M 2210/0618; A61M 2210/0625; A61M 16/201

(57) ABSTRACT

The present disclosure describes an improved endoscopy scope mask and related methods. One such endoscopy mask comprises a mask body defining an interior cavity that fits over a nose and mouth of a patient, in which the mask body comprises a nasal port in fluidic communication with the interior cavity having a silicone membrane that allows for insertion of an endoscopic instrument; an oral port in fluidic communication with the interior cavity having a soft silicone membrane that allows for insertion of the endoscopic instrument; an oxygen port in fluidic communication with the interior cavity; one or more filtered exhalation ports in fluidic communication with the interior cavity; and a suction port in fluidic communication with the interior cavity, the suction port having a connector for attachment to a suction tool. Other embodiments are also provided.

### CROSS-REFERENCE TO RELATED APPLICATION

This application claims priority to co-pending U.S. provisional application entitled, “Droplet Isolation Endoscopy Mask,” having Ser. No. 63/056,099, filed Jul. 24, 2020, which is entirely incorporated herein by reference.

### BACKGROUND

Each year in the United States more than 15 million flexible upper endoscopic procedures are performed according to John's Hopkins. Other sources say this number can exceed 30-35 million when considering all service lines who use flexible scopes including ENT (Ear, Nose, and Throat services), Speech Pathology, Pulmonology, and Plastic Surgery, to name a few. This does not include data on procedures using rigid scopes. To date scoping procedures have been done in clinics with relatively little to no personal protective equipment (PPE). Prior to the Covid-19 pandemic, it is estimated that there is a 13.2% risk of exposure to a patient's body fluids during a GI (gastrointestinal) Endoscope; a 4.1% splash rate to the eyes; and a risk of inhalation of aerosolized microorganisms. Long-term effects have also been identified in endoscopy personnel with reported higher Helicobacter pylori (H. pylori) infection rates which show increased prevalence with consecutive years of practice. With the new outbreak of Covid-19, additional solutions that will help control containment of any airborne diseases are more important than ever.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B show a front facing view of an exemplary endoscopy mask in accordance with various embodiments of the present disclosure.

FIG. 2 shows a representation of the endoscopy mask of FIG. 1A being worn over a person's face.

FIG. 3 shows a prototype of an exemplary endoscopy mask in accordance with various embodiments of the present disclosure.

FIG. 4 shows photographic images of testing prototypes of soft silicone membrane materials for use in the nasal and/or oral ports of an exemplary endoscope mask in accordance with various embodiments of the present disclosure.

FIGS. 5A-5E shows photographic images of various views of a prototype of an endoscopy mask formed of soft silicone materials in accordance with various embodiments of the present disclosure.

FIGS. 6A-6B show photographic images of various views of a prototype of an endoscopy mask formed of soft silicone materials that demonstrates the creation of a nasal port via a punch biopsy tool in accordance with various embodiments of the present disclosure.

### DETAILED DESCRIPTION

Although personal protective equipment (PPE) will continue to have its place, an improved endoscopy scope mask for both oral and nasal scopes is presented within the present disclosure. In one embodiment, such a new mask allows for rigid or flexible scope options along with the additional safety of HEPA (High Efficiency Particulate Air) filtration and in-procedure suctioning for use in the office or operating room.

Current devices on the market allow most types of scopes, probes, and tubes to be inserted through a mask and into the nose or mouth and provides accurate end-tidal CO₂ (Et-CO₂) readings so clinicians can intervene proactively in real time. Although these device provide some degree of protection to the patient and provider during the scope process, they does not filter air from the mask or allow for removal of droplets, secretions, and airborne particles (e.g. virus-sized particles) from the patient wearing the mask.

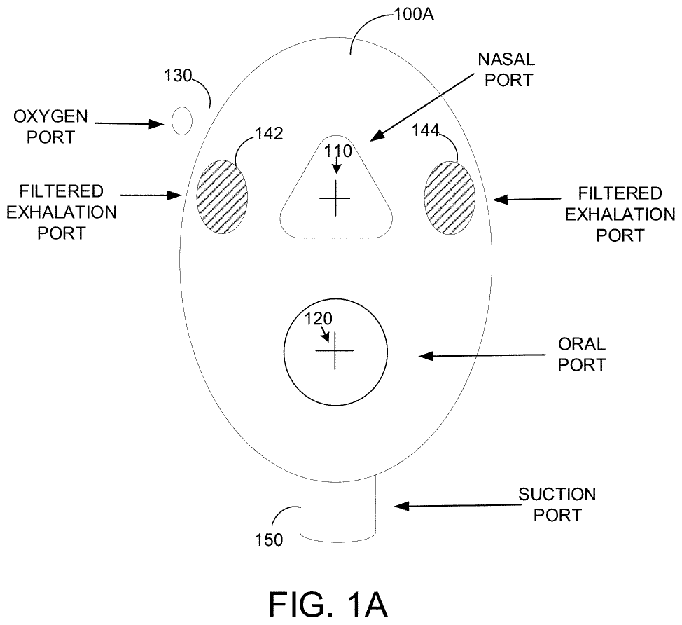

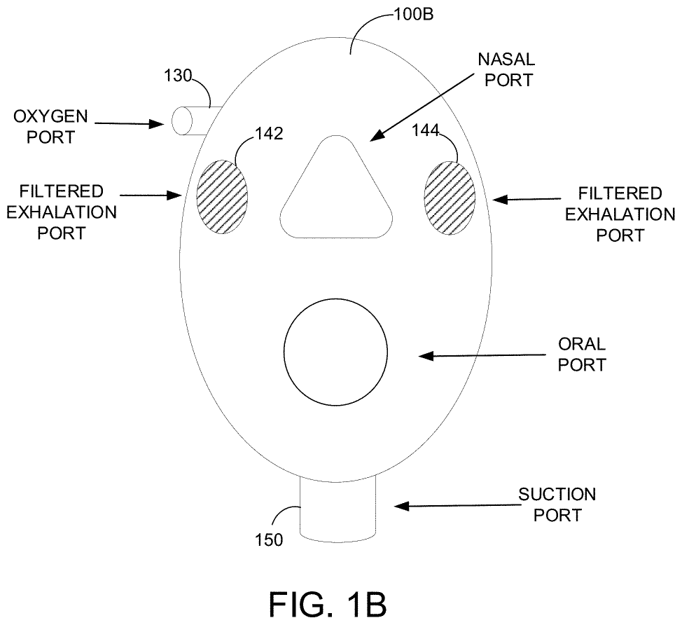

Correspondingly, FIGS. 1A-1B show an exemplary endoscopy mask 100 (e.g., 110A (FIG. 1A), 110B (FIG. 1B)) in accordance with various embodiments of the present disclosure. The mask 100A of FIG. 1A includes a preformed nasal port 110 and/or an oral port 120 to allow for insertion of endoscopic instruments. In various embodiments, the nasal and/or oral ports can be of various shapes, including substantially triangular, circular, or elliptical in shape. Alternatively, the mask 100B of FIG. 1B does not include a preformed nasal port and/or oral port. As such, the nasal port and/or oral port can be created via a punch biopsy tool of similar diameter as the applicable endoscopic instrument. Thus, the punch biopsy tool can be used to puncture a hole in the endoscopy mask 100B such that the applicable endoscopic instrument can be inserted through the hole created by the biopsy punch tool and a seal around the endoscopic instrument would be tight due to the selection of a properly chosen size of the punch biopsy tool and/or the mask material which has elastic properties and will shrink around the inserted endoscopic instrument.

The mask 100 further includes an oxygen port 130 for receiving oxygen or other supplemental gas. In operation, an oxygen delivery tube can be connected to the oxygen port 130. The mask 100 also includes a filtered exhalation port 142, 144 on opposite sides of the mask. In various embodiments, a HEPA (High Efficiency Particulate Air) filter or other type of particulate-removing filter is coupled to the exhalation port 142, 144 to aid in blockage of particulates or droplets from the exhaled air (containing CO₂) of a person wearing the mask 100 from passing through the filter to the environment outside the mask 100, thereby isolating & containing the particulates/droplets to the interior of the mask. Additionally, in various embodiments, the exhalation port 142, 144 may comprise a one-way valve such that air can only leave the mask 100 and cannot enter through the exhalation port 142, 144. To aid in removal of the droplets, secretions, and airborne particles contained within the interior cavity of the mask, a suction port 150 or connector is provided to evacuate these droplets or particles from an interior cavity of the mask 100 using a suction tool. The suction port 150 may also comprise a one-way valve such that air or airborne particles cannot be introduced into the interior cavity of the mask. Correspondingly, the oxygen port may also feature a one-way valve such that particles, secretions, or droplets from an interior of the mask may not exit through the oxygen port to the outside environment.

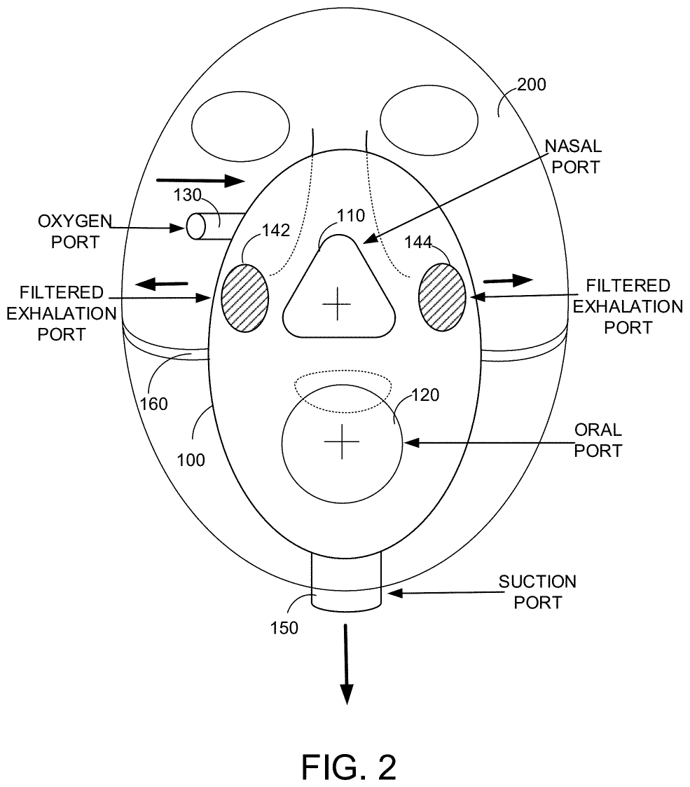

Referring now to FIG. 2, the figure shows a representation of the exemplary endoscopy mask being worn on a person's face 200. Accordingly, in various embodiments, one or more straps 160 may be coupled to a body of the mask 100 and used to fasten the mask around the person's face 200. As shown in the figure, in various embodiments, the mask can cover the person's nose and mouth. As such, an inner portion of the mask 100 defines an interior cavity that will fit over the person's nose and mouth and fit snuggly around at least a portion of the person's face 200. The various ports of the mask are in fluidic communication with the inner cavity and can permit various tasks or functions to perform. As discussed, oxygen can be delivered through the oxygen port 130 to the inner cavity, exhaled air from the patient can be filtered through the exhalation ports 142, 144, endoscopic procedures and/or other procedures (e.g., eating, drinking, administering medication, etc.) can be performed through the nasal and/or oral ports 110, 120, and particles or droplets from within the inner cavity of the mask can be forcibly removed, using suction, via the suction port 150. In various embodiments, the endoscopy mask 100 features soft flexible ports 110, 120 (e.g., self-sealing ports), both orally and nasally, to allow an improved seal around an instrument (e.g., nasal scope, oral scope, etc.) being inserted in the port. The size, angle, and membrane materials (e.g., soft silicone material) for the oral and nasal ports allow for ease in both rigid and flexible scope usage. Correspondingly, the mask 100 itself can be made from one or more of any substances known or used in the art, including but not limited to, soft, polymer materials, including transparent PVC plastic, vinyl, and silicone.

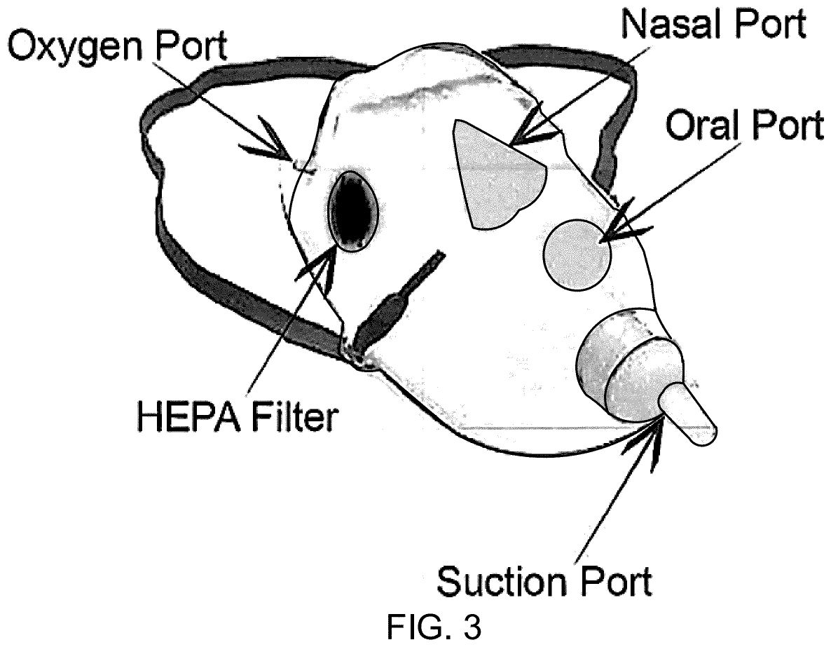

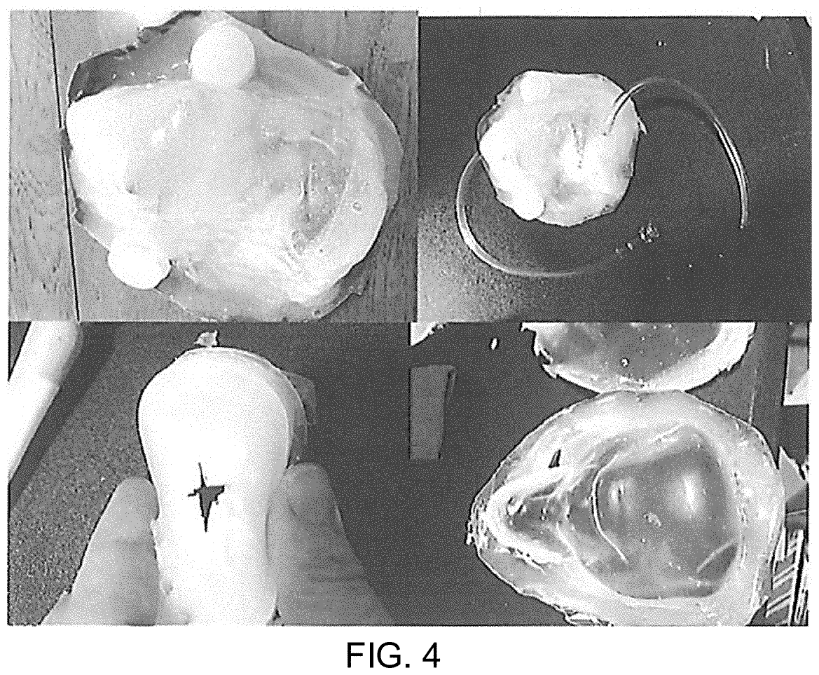

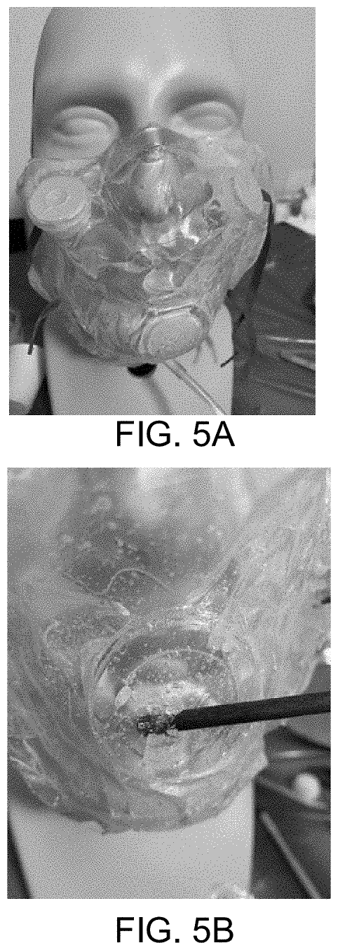

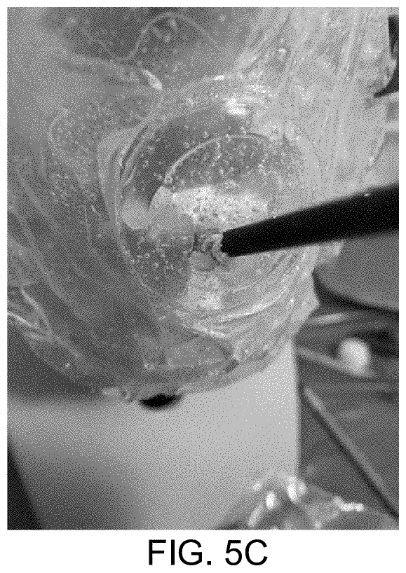

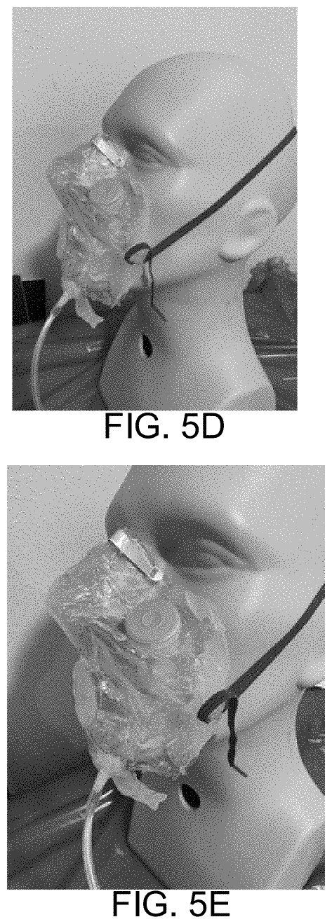

FIG. 3 shows a prototype of an exemplary endoscopy mask in accordance with various embodiments of the present disclosure, in which locations of the various ports are illustrated. FIG. 4 shows photographic images of testing prototypes of soft silicone membrane materials for use in the nasal and/or oral ports. FIGS. 5A-5E show photographic images of various views of a prototype of an endoscopy mask formed of a soft silicone material in accordance with various embodiments of the present disclosure. In particular, FIG. 5A shows a front view of a prototype mask fitted on a mannequin's face, where FIGS. 5B and 5C show close-up views of an endoscopy instrument being inserted within a membrane of the oral port of the mask. Correspondingly, FIG. 5D shows a side view of the prototype mask that is being worn by the mannequin, where FIG. 5E shows a close-up side view of the mask. In accordance with various embodiments, an exemplary endoscopy mask may feature structural contours and curves that match the contours of a person's face, as shown in FIGS. 5A-5B and 5D-5E. Further, in various embodiments, a nose clip may be integrated in the endoscopy mask that can be adjusted to fit the contours of an individual person's face, as shown in FIGS. 5A and 5D-5E.

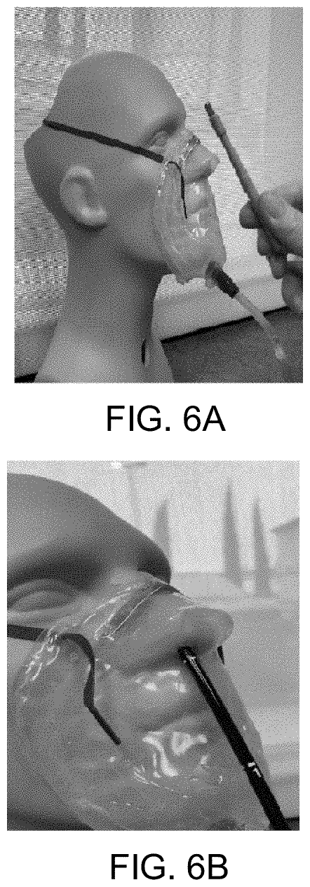

Referring back to FIG. 1B, in various embodiments, a nasal port and/or oral port can be created via a punch biopsy tool of similar diameter as the applicable endoscopic instrument that is to be used with an endoscopy mask 100B. Accordingly, FIGS. 6A-6B show photographic images of views of a prototype of an endoscopy mask formed of a soft silicone material and an exemplary process for creating a nasal port via a punch biopsy tool. Accordingly, FIG. 6A shows a side view of a prototype mask fitted on a mannequin's face, where a punch biopsy tool is shown next to the mask before the tool is to be used to create a nasal port. Correspondingly, FIG. 6B shows a perspective view of the prototype mask being worn by the mannequin as the biopsy tool is inserted into the mask to create a hole for a nasal port. Accordingly, additional ports (e.g., oral port, etc.) can be created within the mask of the appropriate size and location using a punch biopsy tool having a size (e.g., a proper diameter) that is compatible with the endoscopic instrument to be used with the endoscopy mask, in accordance with various embodiments of the present disclosure.

It should be emphasized that the above-described embodiments are merely possible examples of implementations, merely set forth for a clear understanding of the principles of the present disclosure. Many variations and modifications may be made to the above-described embodiment(s) without departing substantially from the principles of the present disclosure. All such modifications and variations are intended to be included herein within the scope of this disclosure.

Therefore, at least the following is claimed:

1. An endoscopy mask comprising:
a mask body defining an interior cavity that fits over a nose and mouth of a patient;
the mask body comprising:
a nasal port in fluidic communication with the interior cavity having a silicone membrane that allows for insertion of an endoscopic instrument;
an oral port in fluidic communication with the interior cavity having a soft silicone membrane that allows for insertion of the endoscopic instrument;
an oxygen port in fluidic communication with the interior cavity;
one or more filtered exhalation ports in fluidic communication with the interior cavity; and
a suction port in fluidic communication with the interior cavity, the suction port having a connector for attachment to a suction tool.

2. The endoscopy mask of claim 1, wherein the one or more filtered exhalation ports are integrated with HEPA filters.

3. The endoscopy mask of claim 1, wherein the one or more filtered exhalation ports comprise a first filtered exhalation port on a first side of the endoscopy mask and a second filtered exhalation port on an opposite side of the endoscopy mask.

4. The endoscopy mask of claim 1, wherein the oxygen port contains a one-way valve that prevents particulates from exiting through the oxygen port.

5. The endoscopy mask of claim 1, wherein the exhalation and suction ports contain one-way valves that prevent particulates from entering through the exhalation and suction ports.

6. The endoscopy mask of claim 1, further comprising at least one strap connected to opposite sides of the endoscopy mask.

7. The endoscopy mask of claim 1, wherein the nasal port is substantially circular in shape.

8. The endoscopy mask of claim 1, wherein the oral port is substantially circular in shape.

9. A method of administering endoscopic procedures to a patient comprising:
securing an endoscopic mask over a nose and mouth of the patient, wherein the endoscopic mask comprises a mask body defining an interior cavity that fits over the nose and mouth of the patient, wherein the mask body comprises:
a nasal port in fluidic communication with the interior cavity having a silicone membrane that allows for insertion of an endoscopic instrument;
an oral port in fluidic communication with the interior cavity having a soft silicone membrane that allows for insertion of the endoscopic instrument;
an oxygen port in fluidic communication with the interior cavity;
one or more filtered exhalation ports in fluidic communication with the interior cavity; and
a suction port in fluidic communication with the interior cavity, the suction port having a connector for attachment to a suction tool;
delivering oxygen to the patient via an oxygen delivery tube to the oxygen port of the endoscopy mask;
administering an endoscopic procedure to the patient through the nasal port or the oral port of the endoscopy mask; and
removing droplets, secretions, or particles introduced by the nose or mouth of the patient via the suction port of the endoscopy mask by applying a suction force via a suction tool coupled to the suction port, wherein the one or more filtered exhalation ports of the endoscopy mask filter and prevent the droplets, secretions, or particles introduced by the nose or mouth of the patient from being passed through the exhalation ports to a surrounding area outside of the endoscopy mask.

10. The method of claim 9, wherein the endoscopic procedure comprises an oral scope.

11. The method of claim 9, wherein the endoscopic procedure comprises a nasal scope.

12. The method of claim 9, wherein the endoscopic procedure is performed in a clinic.

13. A method of administering endoscopic procedures to a patient comprising:
securing an endoscopic mask over a nose and mouth of the patient, wherein the endoscopic mask comprises a mask body defining an interior cavity that fits over the nose and mouth of the patient, wherein the mask body comprises:
an oxygen port in fluidic communication with the interior cavity;
one or more filtered exhalation ports in fluidic communication with the interior cavity; and
a suction port in fluidic communication with the interior cavity, the suction port having a connector for attachment to a suction tool;
punching a nasal port or an oral port within the endoscopic mask using a punch biopsy tool, wherein the punched port is in fluidic communication with the interior cavity and allows for insertion of an endoscopic instrument;
delivering oxygen to the patient via an oxygen delivery tube to the oxygen port of the endoscopy mask;
administering an endoscopic procedure to the patient through the nasal port or the oral port of the endoscopy mask; and
removing droplets, secretions, or particles introduced by the nose or mouth of the patient via the suction port of the endoscopy mask by applying a suction force via a suction tool coupled to the suction port, wherein the one or more filtered exhalation ports of the endoscopy mask filter and prevent the droplets, secretions, or particles introduced by the nose or mouth of the patient from being passed through the exhalation ports to a surrounding area outside of the endoscopy mask.

14. The method of claim 13, wherein both a nasal port and an oral port are punched within the endoscopic mask using the punch biopsy tool.

15. The method of claim 13, wherein an oral port is punched within the endoscopic mask using the punch biopsy tool, wherein the endoscopic procedure comprises an oral scope.

16. The method of claim 15, wherein a nasal port is punched within the endoscopic mask using the punch biopsy tool, wherein the endoscopic procedure comprises a nasal scope.

17. The method of claim 15, wherein the one or more filtered exhalation ports are integrated with HEPA filters.

18. The method of claim 15, wherein the one or more filtered exhalation ports comprise a first filtered exhalation port on a first side of the endoscopy mask and a second filtered exhalation port on an opposite side of the endoscopy mask.

19. The method of claim 15, wherein the oxygen port contains a one-way valve that prevents particulates from exiting through the oxygen port.

20. The method of claim 15, wherein the exhalation and suction ports contain one-way valves that prevent particulates from entering through the exhalation and suction ports.
